(19) United States
(12) Patent Application Publication
(10) Pub. No.: US 2016/0175207 A1
(43) Pub. Date: Jun. 23, 2016

(54) TEETH WHITENING FILM UTILIZING TRIPOLYPHOSPHATE SALTS

(71) Applicant: Steven D. Jensen, West Jordan, UT (US)

(72) Inventor: Steven D. Jensen, West Jordan, UT (US)

(21) Appl. No.: 14/971,640

(22) Filed: Dec. 16, 2015

Related U.S. Application Data
(60) Provisional application No. 62/093,085, filed on Dec. 17, 2014.

(51) Int. Cl. A61K 8/24; A61K 8/02; A61Q 11/00; A61K 8/42

(52) U.S. Cl. CPC A61K 8/24; A61K 8/42; A61K 8/0233; A61Q 11/00; A61K 2800/805; A61K 2800/596

(57) ABSTRACT

A teeth whitening film may include a water insoluble polymeric sheet. A gelatinous active layer may be located on a surface of the water insoluble polymeric sheet. The gelatinous active layer may include a tripolyphosphate salt.

### CROSS-REFERENCES TO RELATED APPLICATIONS

This application claims the benefit of U.S. Provisional Application No. 62/093,085 filed on Dec. 17, 2014, which is hereby incorporated by reference in its entirety.

### TECHNICAL FIELD OF THE INVENTION

The present invention relates to the field of dentistry and, more particularly, devices useful for cleaning and whitening teeth.

### SUMMARY OF THE INVENTION

Teeth whitening devices, methods of making such teeth whitening devices, and methods of using such teeth whitening devices are described herein.

In one embodiment, a teeth whitening film may include a water insoluble polymeric sheet. A gelatinous active layer may be located on a surface of the water insoluble polymeric sheet. The gelatinous active layer may include a tripolyphosphate salt.

Additional features and advantages of the invention will be set forth in the description which follows, and in part will be obvious from the description, or may be learned by the practice of the invention. The features and advantages of the invention may be realized and obtained by means of the instruments and combinations particularly pointed out in the appended claims. These and other features of the present invention will become more fully apparent from the following description and appended claims, or may be learned by the practice of the invention as set forth hereinafter.

### BRIEF DESCRIPTION OF THE DRAWINGS

In order to describe the manner in which the above-recited and other advantages and features of the invention can be obtained, a more particular description of the invention briefly described above will be rendered by reference to specific example embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings.

FIG. 1 depicts a perspective view of a whitening film according to an embodiment of the present invention.

FIG. 2 depicts a side view detail of the whitening film of FIG. 1.

### DETAILED DESCRIPTION

Aqueous tripolyphosphate salts whiten teeth by dissolving susceptible stains into solution. The average person brushes their teeth for less than 60 seconds; therefore the contact time of a tripolyphosphate salt delivered via a toothpaste is limited on the teeth. Embodiments of the present invention provide delivery methods that allow greater contact time for a greater stain dissolution effect upon the teeth.

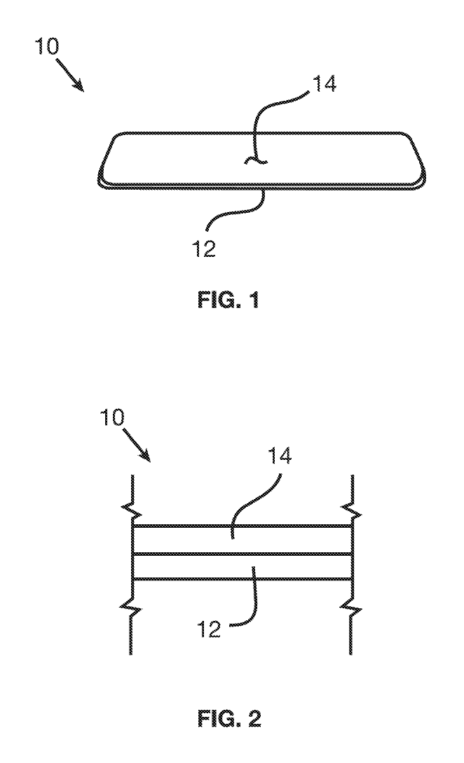

As shown in FIGS. 1 and 2, a whitening film 10 according to an embodiment of the present invention utilizes a thin water insoluble polymeric or plastic flexible backing material 12 to which is applied a water soluble gelatinous layer 14 that contains the active ingredient tripolyphosphate salts. The finished whitening film 10 is applied to the teeth such that the water soluble gelatinous layer 14 including the active ingredient comes into contact with the tooth surface. The water insoluble backing material 12 faces away from the teeth and functions to protect the gelatinous layer 14 from the dilution effects of saliva. The finished whitening film 10 is designed to be worn for more than one minute; other embodiments call for treatment times comprising: 15, 30, 60, 120, 180, 240, 360 minutes and any other suitable application time. An overnight whitening film also comprises an embodiment of the invention, wherein the user wears the whitening film while they sleep. The user can create a stain removal regime lasting from days to weeks to achieve a desired whiteness; the film is disposable and can be replaced multiple times per day if desired.

Embodiments of the thin polymeric or plastic backing material 12 include but are not limited to: polyethylene sheet, polypropylene sheet, polyurethane sheet, nylon sheet, wax sheet, parafilm sheet, PVC sheet, and any other useful water insoluble sheet material.

The gelatinous composition 14 that contains the active ingredient may include a liquid component, a polymeric thickener, and a component that adjusts pH. The gelatinous composition that contains the active ingredient may also include additional components such as a sweetener, flavoring agent, fluoride, anti-tartar agent, anti-microbial, and any other useful ingredient.

Embodiments of thickening agents include but are not limited to: PVP, polyethyl oxazoline, carboxypolymethylene, guar gum, xanthum gum, silica powder, fumed silica, polyethylene glycol, polypropylene glycol, and any other useful thickening agent.

Embodiments of liquid components include but are not limited to: water, ethanol, propylene glycol, glycerin, propanol, and any other useful liquid component.

Embodiments of pH adjustment compound include but are not limited to: potassium hydroxide, sodium hydroxide, hydrochloric acid, sulfuric acid, phosphoric acid and any other pH adjustment medium.

The salts of tripolyphosphate include but are not limited to: sodium tripolyphosphate and potassium tripolyphosphate.

The components are blended together until a homogenous gel is created usually by mixing with a mechanical mixer. The gel is spread as a thin layer over the water-insoluble polymeric backing material 12 and is allowed to dry into a gelatinous state, forming the gelatinous composition 14. The final whitening film 10 is cut into a size to fit at least some portion of the teeth and packaged in airtight containers.

A “gelatinous” material as used herein has physical properties somewhat similar to the confection known as gummy worms (i.e., flexible and soft enough to conform to a tooth surface without cracking or fracturing, yet firm enough that it will not coalesce with a separate gelatinous material when placed into mutual contact).

Another embodiment of the invention combines the stain removing ability of tripolyphosphate with the whitening/oxidation ability of peroxide into the gelatinous composition 14. To our surprise we have found the sodium tripolyphosphate to be compatible with peroxide and the two can remain soluble together in solution. Therefore any combination of peroxide and sodium/potassium tripolyphosphate is within the scope of this patent. The ideal combination would maximize the concentration of both ingredients with respect to their solubility within the polymeric thickener matrix.

### EXAMPLE #1

10%—sodium tripolyphosphate
30%—poly 2-ethyl oxazoline
10%—ethanol
0.3%—peppermint oil
0.2%—sucralose
49.5%—water

The components are blended together until homogenous in a mechanical mixer. The resulting gel is then spread as a thin layer over a water-insoluble polymeric backing material, such as parafilm, and dried until gelatinous.

### EXAMPLE #2

10%—potassium tripolyphosphate
30%—PVP
5%—polyethylene glycol 35,000 m.w.
0.3%—peppermint oil
0.2%—sucralose
54.5%—water

The components are blended together until homogenous in a mechanical mixer. The resulting gel is then spread as a thin layer over a water-insoluble polymeric backing material such as polypropylene sheet and dried until gelatinous.

### EXAMPLE #3

5%—potassium tripolyphosphate
28%—PVP
5%—polyethylene glycol 35,000 m.w.
0.3%—peppermint oil
0.2%—sucralose
36.5%—water
10%—carbamide peroxide
15%—ethanol

The components are blended together until homogenous in a mechanical mixer. The resulting gel is then spread as a thin layer over a water-insoluble polymeric backing material such as polypropylene sheet and dried until gelatinous.

While certain embodiments and details have been included herein and in the attached invention disclosure for purposes of illustrating the invention, it will be apparent to those skilled in the art that various changes in the methods and apparatuses disclosed herein may be made without departing from the scope of the invention, which is defined in the appended claims.

What is claimed is:

1. A teeth whitening film comprising:
a water insoluble polymeric sheet; and
a gelatinous active layer located on a surface of the water insoluble polymeric sheet, the gelatinous active layer comprising a tripolyphosphate salt.

2. The teeth whitening film of claim 1, wherein the gelatinous active layer further comprises a peroxide.

3. A method of making a teeth whitening film, the method comprising:
positioning a water soluble gel that comprises a tripolyphosphate salt on a water insoluble polymeric sheet; and
drying the water soluble gel to a gelatinous state.
